(19) United States
(12) Patent Application Publication
(10) Pub. No.: US 2025/0388957 A1
(43) Pub. Date: Dec. 25, 2025

(54) DEMONSTRATING FETAL DNA IN THE MATERNAL BREATH

(71) Applicants: Canan UNAL, Ankara (TR); Mehmet Sinan BEKSAC, Ankara (TR); Sitki TIPLAMAZ, Istanbul (TR); Mustafa AKKIPRIK, Istanbul (TR); Irem PEKER EYUBOGLU, Istanbul (TR)

(72) Inventors: Canan UNAL, Ankara (TR); Mehmet Sinan BEKSAC, Ankara (TR); Sitki TIPLAMAZ, Istanbul (TR); Mustafa AKKIPRIK, Istanbul (TR); Irem PEKER EYUBOGLU, Istanbul (TR)

(21) Appl. No.: 18/879,266

(22) PCT Filed: Jun. 26, 2023
(86) PCT No.: PCT/TR2023/050628

§ 371 Date: Dec. 27, 2024

(30) Foreign Application Priority Data
Jun. 27, 2022 (TR) 2022/010620

(51) Int. Cl. C12Q 1/686; C12Q 1/6881; C12Q 1/6886

(52) U.S. Cl. CPC C12Q 1/686; C12Q 1/6881; C12Q 1/6886

(57) ABSTRACT

A method of detecting fetal cell-free fetal DNA (cffDNA) in the breath of the pregnant woman is provided. The method includes condensation of the breath sample to liquefy; selection of the target genome region locus; selection of primers that amplify the selected locus; selection of probes that attach to the replicated locus; introducing the targeted site primers and the breath sample into the PCR cycle and amplifying the targeted locus, if present in the sample; binding the probes specific to the amplified region; detecting the fluorescent radiation with the PCR reader and presenting it as data, if there is a targeted region in the breath sample.

### CROSS REFERENCE TO THE RELATED APPLICATIONS

This application is the national phase entry of International Application No. PCT/TR2023/050628, filed on Jun. 26, 2023, which is based upon and claims priority to Turkish Patent Application No. 2022/010620, filed on Jun. 27, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBUY185_Sequence_Listing.xml, created on Dec. 19, 2024, and is 3,482 bytes in size.

### TECHNICAL FIELD

The present invention relates to a method of displaying fetal DNA in the maternal breath. The invention has the potential to be widely used in the fields of health and livestock.

### BACKGROUND

In the routine follow-up of pregnancy, a number of tests and examinations such as combined tests and triple tests are performed for genetic anomalies that are likely to be present in the fetus. These follow-ups are made with anamnesis, abdominal ultrasonography (usg) examinations, and blood tests. Pregnant women with a high risk of genetic anomaly in the fetus according to the results of these tests and examinations, are referred to advanced tests such as chorionic villus biopsy, amniocentesis, or blood cell-free Fetal DNA (cffDNA) analysis. Chorionic villus biopsy is performed by entering from the vagina with a probe and taking a biopsy sample from the placenta under the guidance of abdominal USG and this sample is subjected to genetic analysis. There is a 1-3% risk of miscarriage in this procedure. Amniocentesis, on the other hand, is again guided by abdominal USG and the probe is inserted into the gestational sac and the fluid is withdrawn from there. There is a 0.5-1% risk of miscarriage in this procedure. Both of the above-mentioned procedures are invasive and are uncomfortable for the pregnant woman and risks the life of the fetus. A newer method than these methods is cffDNA analysis. cffDNA is fetal DNA freely circulating in maternal blood. Maternal blood is sampled by venipuncture. cffDNA analysis is called the non-invasive prenatal screening method requested for pregnant women of advanced maternal age. Although it is called a non-invasive method, venous blood collection is also an invasive procedure.

Cell-free DNA (cfDNA) and cell-free fetal DNA (cffDNA) are different from each other. In pregnant women, cfDNA refers to maternal DNA, while cffDNA only refers to fetal DNA. Although cell-free DNA (cfDNA) is known to be in all body fluids such as sweat, urea, CSF, breath, cell-free fetal DNA (cffDNA) has not been demonstrated in breath until now. In the known state of the technique, DNA analyses of the cells that the breath comes into contact with as it exits the body (DNA from cells of the respiratory and gastrointestinal systems and DNA from microorganisms in the microbiota) have been conducted. In the document numbered U.S. Pat. No. 10,975,431 B2, an algorithm was created regarding the stage of cancer according to fragmentation rate by looking at certain gene regions (tumour suppressor or tumour control gene regions) only in cfDNAs obtained from plasma. In order to carry out the above-mentioned procedure, the diagnosis of the person must be made by another method beforehand. People who are not sick also have these regions and these can be amplified, the general tumour control regions were amplified and the fragmentation rate was checked, without directly targeting the cancer cell-specific genetic region, a result was tried to be obtained from the fragmentation rate in the above-mentioned patent. Therefore, an attempt has been made to diagnose indirectly. For this reason, created algorithms cannot diagnose tumours early enough and cannot clearly differentiate tumour type (lung small cell-multiple myeloma). Moreover, its use as a prenatal test is not mentioned. In addition, bloodletting is required to obtain plasma, and bloodletting is an invasive procedure.

In the patent document numbered WO 2013/116503 A2, it is mentioned that by amplifying miRNAs from condensed breath by RT-PCR or by amplifying the pathogen genome (DNA/RNA), only diseases involving the lung can be diagnosed. In the invention, droplet digital (ddPCR) was used and fetal DNA that originated from the urogenital system was amplified, where the breath does not come into direct contact, this shows that it is possible to screen nucleic acids belonging to cell groups that do not come into direct contact with breath from the breath (e.g., detecting DNA regions specific to prostate cancer from breath or detecting DNA of transplant kidney in breath in kidney transplanted patient), therefore, the invention is completely different from the relevant document.

In the known state of the technique; DNA belonging to cells that the breath does not come into direct contact with (except respiratory and gastrointestinal system cells and their microbiota) could not be demonstrated in condensed breath.

### SUMMARY

The primary object of the present invention is to show the DNA of the mass (fetus) in a region (in the urogenital system) that does not have direct contact with the breath is exhaled, with a non-invasive method. It has been shown that condensed breath does not only exemplify the respiratory and gastrointestinal system but also the whole body system by showing the presence of fetal DNA in the condensed breath of pregnant women with the present invention. In the studies carried out up to the present, DNA analyses of the cells (DNA of the cells of the respiratory and gastrointestinal system and of the living things belonging to the microbiota) that the breath comes into contact with while leaving the body have been studied. In this invention, the quantification was made by showing for the first time that the DNA of the mass (fetus) in a region that does not have direct contact with the breath (in the urogenital system) is exhaled. In the ddPCR method, if the nucleic acid is detected in the studied sample, its amount is automatically determined, not as in real-time PCR which needs to be compared with samples with known nucleic acid content. Quantification helps to understand the relationship between the amount of nucleic acid (RNA/DNA) obtained in the breath and other factors (e.g., the relationship between the weight of the fetus and the body mass index of the pregnant woman and the amount of fetal DNA detected in the breath).

It has been shown that the DNA of the fetus in the mother's womb can be amplified by a completely non-invasive method with the present invention. In the future, it is predicted that DNA belonging to cancer and transplanted organs can be obtained with this method.

In the method of the present invention, the breath exhaled with routine breathing is cooled and condensed and the resulting liquid is genetically analyzed, this method is completely non-invasive and does not harm the pregnant woman.

It was also quantified with the present invention by showing for the first time that the DNA of the mass (fetus) in another region (urogenital system) that does not have direct contact with the breath is in the breath. In other words, it has been shown for the first time that breath can genetically sample the whole body.

This invention provides the opportunity to diagnose the person according to whether the DNA specific to the tissue mass (tissues other than respiratory and gastrointestinal systems, e.g.: urogenital system tissue-fetus) that the breath does not directly contact with is directly amplified or not by obtained exhaled breath condensate.

It has been shown with the invention that by amplifying a Y chromosome sequence directly in the breath in a pregnant woman carrying a male fetus, it is possible to diagnose that she is carrying a male fetus just by condensed breath. This invention also showed that the DNA of cells belonging to different systems can be detected in breath. It is predicted that in the future, other fetal genetic anomalies and cancers can be detected with this method, apart from sex determination.

The method introduced by the invention is a completely non-invasive method as it is performed only by condensing the breath.

Y chromosome locus amplification in the condensed breath (EBC) of the pregnant woman was examined. It was determined that there was amplification in pregnant women carrying a male fetus, and no amplification occurred in pregnant women carrying a female fetus (FIG. 1 and FIG. 2).

Obtaining fetal DNA in the breath of the pregnant woman means that the DNA of the masses (tumours, transplanted organs etc.) formed in the body can be exhaled, apart from the respiratory and gastrointestinal tract. For this reason, it is foreseen that in the near future, with this method, genetic analysis of condensed breath, screenings related to both fetuses and cancers other than respiratory and gastrointestinal systems can be performed.

### BRIEF DESCRIPTION OF THE DRAWINGS

The figures and related explanations necessary for a better understanding of the subject of the invention are as follows.

FIG. 1: Points above the threshold represent radiant droplets and indicate DNA amplification. Points below the threshold represent non-radiating droplets and mean that there is no DNA amplification. Example 3 and Example 9 are condensed breath samples from women bearing male fetuses, and the Blank sample is a sample that does not contain any DNA.

FIG. 2: Bar graph of ddPCR results of condensed breath samples; the X-axis gives the number of fluorescent droplets. Samples with NTC (No template control) do not contain any DNA, the threshold value was calculated as 6. N3 and N9 samples, which radiate droplets above this value, are seen as positive and other samples as negative. Both samples were obtained from the woman carrying a male fetus.

### DETAILED DESCRIPTION OF THE EMBODIMENTS

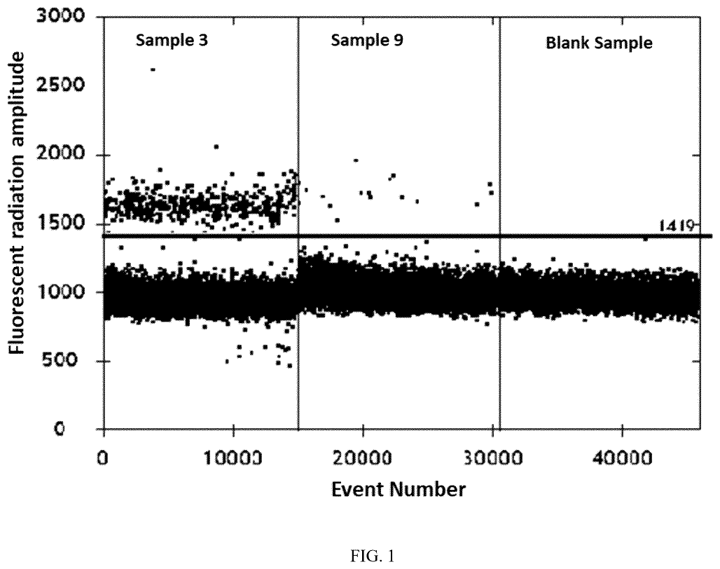

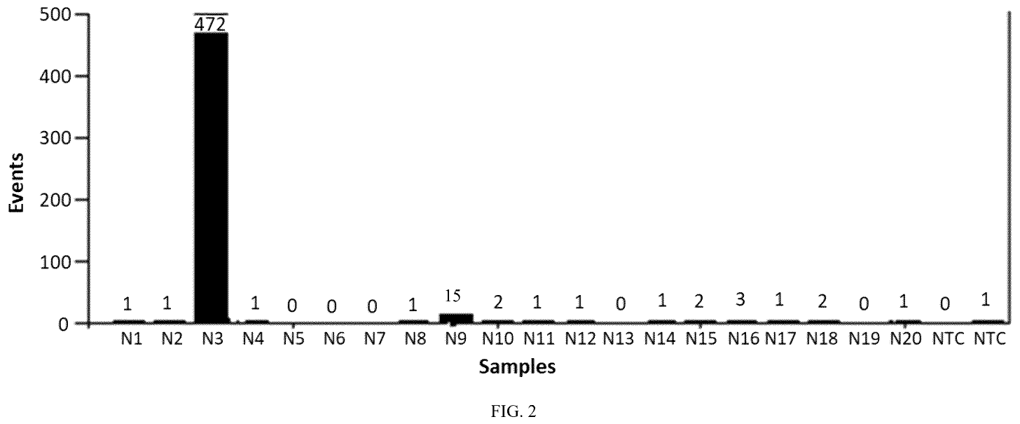

It is known that trophoblasts, which constitute the majority of fetal cells participating in maternal blood circulation, are eliminated by the maternal lungs. It is also known that cell-free DNA (cfDNA) is detected in other body fluids such as sweat, urea, and CSF. Y chromosome locus amplification in the condensed breath (EBC) of the pregnant woman was studied in the present invention based on this information. It was determined that there was amplification in pregnant women carrying a male fetus, and no amplification occurred in pregnant women carrying a female fetus (FIG. 1 and FIG. 2).

No study to date has investigated the presence of fetal DNA from condensed breath. This method has the potential to be the beginning of subsequent fetal DNA analysis, and even to be used not only in fetal DNA analysis but also in cancer screening and transplanted organ follow-ups.

Method of detecting the DNA of the tissue mass in a region that is not in direct contact with the breath in the condensed breath, comprises the following process steps;
a. Condensation of the breath sample to liquefy,
b. Selection of the target genome region locus,
c. Selection of primers that amplify the selected locus,
d. Selection of probes that attach to the replicated locus,
e. Introducing the targeted site primers and the breath sample into the PCR cycle and amplifying the targeted locus, if present in the sample,
f. Binding the probes selected in step d specific to the amplified region,
g. Detecting the fluorescent radiation with the PCR reader and presenting it as data, if there is a targeted region in the examined breath sample.

The method of detecting cell-free fetal DNA (cffDNA) of the fetus originating from the urogenital system in the mother's breath, comprises the following process steps;
a. Condensation of the pregnant mother's breath sample to liquefy,
b. Selecting the DYS14 locus on the Y chromosome as the target genome region,
c. Selecting DYS14-F1 5′ GGGCCAATGTTGTATCCTTCTC-3′ (SEQ ID NO: 1), DYS14-R1 5′-GCCCATCGGTCACTTACACTTC-3′ (SEQ ID NO: 2) that amplify the 84 bp region as primers,
d. Selecting DYS14-Taqman probe 5′-TCTAGTGGAGAGGTGCTC-3′ (SEQ ID NO: 3) as probe,
e. Introducing primers of the targeted region into the PCR cycle in ddPCR and amplifying the targeted locus if it exists in the sample,
f. Binding the probes selected in step d specific to the amplified region,
g. Detecting the fluorescent radiation with the ddPCR reader and presenting it as data, if there is a targeted region in the examined sample.

The procedure steps for detecting fetal DNA in the mother's breath are detailed below.
1—The pregnant participant, who is asked to stop eating and drinking at least 1 hour in advance (except for drinking water), is asked to rinse her mouth thoroughly with sterile water before the condensed breath sample is taken. The participant is asked to breathe normally (tidal) for 10 minutes and liquefied by condensation with the EcoScreen™ device (Jaeger, Wurzburg, Germany). Other commercial instruments can be used instead of this device.
2—The resulting liquid is divided into 500 μL aliquots and stored first at −21° C. and then at −80° C. until the next processing.
3—400 μL of condensed exhaled breath liquid is condensed to 40 μL of DNA using the DSP Virus Kit (Qiagen, Hilden, Germany) according to the manufacturer's instructions. Other commercial kits can be used instead of this kit. Each frozen aliquot is thawed at most once.
4—The DYS14 locus on the Y chromosome was chosen as the target genome region, since this locus is more likely to be detected as it is repeated multiple times on the Y chromosome. DYS14-F1 5′-GGGCCAATGTTGTATCCTTCTC-3′ (SEQ ID NO: 1), DYS14-R1 5′-GCCCATCGGTCACTTACACTTC-3′ (SEQ ID NO: 2) that amplify the 84 bp were selected region as primers. DYS14-Taqman probe 5′-TCTAGTGGAGAGGTGCTC-3′ (SEQ ID NO: 3) is used as probe. These primers were used to amplify a region of the DYS14 locus, and then the amplified region was marked with a specific probe to give fluorescence. These target loci can be changed, and even different genetic regions can be used as targets by taking advantage of methylation differences. This raises the possibility of performing cancer and fetal genetic screenings with this invention in the future.
5—Targeted DNA was detected and quantified with the QX200™ Droplet Digital™ PCR system (Bio-Rad Laboratories, Hercules, CA, USA). Briefly, 11 μL of 2×ddPCR supermix (Bio-Rad Laboratories) is mixed with 0.8 μL of each primer, 0.4 μL Taqman probe, 8 μL of prepared sample so that the reaction volume is 22 μL. 70 μL of droplet forming oil (Bio-Rad Laboratories) is added to the 20 μL ddPCR reaction volume so as to form droplets and placed in the automated droplet generator (Bio-Rad Laboratories) according to the manufacturer's instructions. 40 μL of the formed droplets are drawn and placed in a 96-well PCR plate (Eppendorf, Hamburg, Germany) and the targeted DNA locus is amplified with the C1000 Touch™ Thermal cycler (Bio-Rad Laboratories). Amplification is initiated by activating DNA polymerase by incubation at 95° C. for 10 minutes, then 40 cycles; denaturation is set at 94° C. for 30 seconds, it is kept at 60° C. for 1 minute for annealing and elongation, finally it is held at 98° C. for 10 minutes for enzyme inactivation. After this thermal cycle, the plate is placed in the QX200™ Droplet Reader (Bio-Rad Labs). The amount of copies/μL of DNA targeted with the Bio-Rad Laboratories system QuantaSoft 1.3.2.0 is calculated using Poisson distribution analysis. The same or more sensitive PCR methods (such as BEAMing) can be used instead of this PCR method.

When it is demonstrated that the Y chromosome locus is amplified in maternal breath samples, it is determined that the pregnant woman is carrying a male fetus.

What is claimed is:

1. A method of detecting DNA of a tissue mass in a region, wherein the region is not in direct contact with a breath in a condensed breath, the method comprises the following process steps;
a. condensation of a breath sample to liquefy,
b. selection of a target genome region locus,
c. selection of primers, wherein the primers amplify the target genome region locus,
d. selection of a probe, wherein the probe attaches to a replicated locus,
e. adding the primers and the breath sample into a PCR cycle, and amplifying the target genome region locus when the target genome region locus is present in the breath sample,
f. binding the probe selected in the step d specific to an amplified region, and
g. detecting a fluorescent radiation with a PCR reader and presenting as data when a targeted region is present in the breath sample.

2. The method according to claim 1, wherein the PCR cycle in the step e is a droplet digital (ddPCR) cycle.

3. The method according to claim 1, wherein the tissue mass is a tumour, a transplanted organ, or a fetus.

4. The method according to claim 3, wherein a detection of fetal cell-free fetal DNA (cffDNA) in a urogenital system, in a mother's breath comprises the following process steps;
a. condensation of a pregnant mother's breath sample to liquefy,
b. selecting a DYS14 locus on a Y chromosome as the target genome region locus,
c. selecting DYS14-F1 5′ GGGCCAATGTTGTATCCTTCTC-3′ (SEQ ID NO: 1) and DYS14-R1 5′-GCCCATCGGTCACTTACACTTC-3′ (SEQ ID NO: 2) as the primers, wherein the primers amplify an 84 bp region,
d. selecting DYS14-Taqman probe 5′-TCTAGTGGAGAGGTGCTC-3′ (SEQ ID NO: 3) as the probe,
e. adding the primers into the PCR cycle in ddPCR, and amplifying the target genome region locus when the target genome region locus is present in the pregnant mother's breath sample,
f. binding the probe selected in the step d specific to amplified region, and
g. detecting the fluorescent radiation with a ddPCR reader and presenting as the data when the targeted region is present in the pregnant mother's breath sample.
